(19) United States
(12) Patent Application Publication
(10) Pub. No.: US 2001/0046685 A1
(43) Pub. Date: Nov. 29, 2001

(54) Control for methods for determining platelet count and platelet function

(76) Inventors: Keith A. Moskowitz, San Diego, CA (US); Victor A. Manneh, San Diego, CA (US); Boris I. Ratnikov, San Diego, CA (US)

Correspondence Address: William Schmonsees, Heller Ehrman White & McAuliffe LLP, 275 Middlefield Road, Menlo Park, CA, 94025-3506, US

(21) Appl. No.: 09/895,776

(22) Filed: Jun. 28, 2001

Related U.S. Application Data
(63) Continuation of application No. 09/321,673, filed on May 28, 1999, now abandoned.

(51) Int. Cl. G01N 33/567

(52) U.S. Cl. 435/7.21

(57) ABSTRACT

The present invention concerns a composition comprising an aqueous medium, a reagent for assessing fibrinogen biological activity and a reagent for assessing the activity of a reagent used for determining platelet count. In one particular embodiment the composition comprises an aqueous medium, an antibody for fibrinogen and fixed platelets substantially free of fibrinogen antibody binding sites. Also disclosed is a method for conducting a control for an assay for platelet function activity and a control for the platelet count assay. The method comprises utilizing a common control for the assays wherein the control does not cross-react with itself or with reagents for conducting the assays. In a particular embodiment the common control comprises an aqueous medium, a reagent for assessing fibrinogen biological activity and a reagent for binding to the reagent used for determining platelet count. Also disclosed are kits for carrying out methods in accordance with the present invention.

### BACKGROUND OF THE INVENTION

1. Field of the Invention

This invention relates to the field of diagnostic assays and in particular to the determination of platelet function activity and the number of platelets in a sample.

The role of platelets in mammalian physiology is extraordinarily diverse, but their primary role is in promoting hemostasis. In many situations, an evaluation of the ability of blood to clot is desired, a parameter that is frequently controlled by the ability of platelets to adhere and/or aggregate. Of interest, therefore, is the assessment of the adhesive functions of platelets. For example, questions of interest include whether to administer drugs that will block, or promote, clot formation, or whether to detect deficiencies in platelet function prior to surgical procedures. Also of interest is evaluating the effectiveness of a platelet inhibitor that is being tested as a new drug or is being used as an approved clinical treatment in a patient.

Platelets are known to aggregate under a variety of conditions and in the presence of a number of different reagents. Platelet aggregation is a term used to describe the binding of platelets to one another. The phenomenon can be induced by adding aggregation inducing agents to platelet rich plasma (PRP) or to whole blood. Platelet aggregation in vitro depends upon the ability of platelets to bind fibrinogen to their sufaces after activation by an aggregation inducing agent such as ADP or collagen.

Platelets play a critical role in the maintenance of normal hemostasis. When exposed to a damaged blood vessel, platelets will adhere to exposed sub endothelial matrix. Following the initial adhesion, various factors released at the site of injury, such as thrombin, ADP and collagen, activate the platelets. Once platelets are activated, a conformational change occurs in the platelet glycoprotein GPIIb/IIIa receptor allowing it to bind fibrinogen and/or von Willebrand factor.

It is this binding of the multivalent fibrinogen and/or von Willebrand factor molecules by GPIIb/IIIa receptors on adjacent platelets that results in the recruitment of additional platelets to the site of injury and their aggregation to form a hemostatic plug or thrombus.

In vitro platelet aggregation is the laboratory method used to assess the in vivo ability of platelets to form the aggregates leading to a primary hemostatic plug. In this technique an aggregating agent such as ADP or collagen is added to whole blood or PRP and aggregation of platelets monitored. Platelet aggregometry is a diagnostic tool that can provide insights difficult to obtain by other techniques, thus aiding in patient diagnosis and selection of therapy. These methods of monitoring platelet aggregation require expensive, dedicated laboratory instruments that are not easily portable and require standardization to ensure accurate quantitative results. In addition, unless performed using whole blood, results are unlikely to be available for several hours.

Currently there are two detection methods used in instruments with FDA clearance for performing platelet aggregometry: optical and impedance measurements. The CHRONO LOG Model 530 and Model 540 use the optical method for PRP and the impedance method for whole blood aggregometry. The impedance method has been shown to be substantially equivalent to the optical method for measuring platelet aggregation in PRP.

A rapid platelet function assay has recently been developed and is described in U.S. Pat. No. 5,763,199 (Coller). The assay determines glycoprotein (GP) lIb/IlIa receptor blockade in whole blood. Agglutination of small polymeric beads coated with a GPIIb/IIIa ligand such as fibrinogen results when the beads are contacted with whole blood containing platelets with GPIIb/IIIa receptors that are not blocked. Failure to agglutinate indicates that blockade of the GPIIb/IIIa receptors has been achieved. In a preferred embodiment, the addition of a thrombin receptor activator results in an assay that is rapid and convenient enough to be performed at bedside and that results in agglutination of the small polymeric beads within a convenient, known period of time if the GPIIb/IIIa receptors are not blocked. The assay includes the ability to transfer blood to be tested from a collection container to an assay device without opening the collection container. This platelet aggregation assay can be conducted at the same time as the activated clotting time (ACT), which is performed to assess the adequacy of heparinization. During chronic infusions of GPIIb/IIIa antagonists, or with chronic oral therapy, periodic monitoring may also be desirable. In certain circumstances, as for example, prior to surgery or an invasive procedure, it may be desirable to rapidly determine whether the effect of the GPIIb/IIIa antagonist has worn off sufficiently to allow the surgery or procedure to be performed without further interventions to reverse the effect of the GPIIb/IIIa inhibitor. Finally, in the event of bleeding complications, a rapid measure of platelet function may be helpful in determining whether the bleeding is due to a high or toxic level of platelet inhibition. The level of platelet inhibition may also be helpful in guiding whether to reverse the drug effect with platelet transfusions or look for other causes of bleeding.

In the performance of the above assay it is desirable to know whether a low result in the platelet function assay is due to low platelet function activity or simply to a low number of platelets. There are a number of approaches currently used to assess platelet number. For example, the number of platelets in a plasma sample may be determined by electronic particle enumeration using, for example, an instrument manufactured by Coulter Electronics (Hialeah, Fla.). In another approach a whole blood analyzer such as, for example, an instrument manufactured by Sysmex Corporation of America (Long Grove, Ill.), is used. This instrument measures all cells in the whole blood.

In using the platelet function assay mentioned above, it is desirable to carry out both the platelet function assay and a platelet count measurement in the same instrument. This avoids time delays in using the above instruments for platelet count measurement, which typically may not be present at the site of the platelet function assay, for example, a patient's bedside. It is further desirable to have a combined or common control for both the platelet function assay and the platelet count assay. Ideally, the reagent for each assay control should not crossreact with other assay reagents. Thus, the reagents for the control for the platelet count assay should not interact with the reagents for the platelet function assay and vice versa and the reagents for both controls should not interact with each other when they are combined.

2. Previous Disclosures

A rapid platelet function assay is described in U.S. Pat. No. 5,763,199 (Coller).

Methods for determing platelet count are described in U.S. Pat. application Ser. No. 09/177,884 filed Oct. 23, 1998 (Moskowitz and Coller)

Agglutrimetric assays in blood are described in U.S. Pat. application Ser. No. 08/820,999 filed Mar. 20, 1997 (Durbin, et al.).

### SUMMARY OF THE INVENTION

One embodiment of the present invention concerns a composition comprising an aqueous medium, a reagent for assessing fibrinogen biological activity and a reagent for assessing activity of the reagent for determining platelet count.

Another embodiment of the present invention is a composition comprising an aqueous medium, an antibody for fibrinogen and fixed platelets substantially free of fibrinogen antibody binding sites.

Another embodiment of the present invention is a method for conducting a control for an assay for platelet function activity and a control for an assay for platelet count, the method comprising utilizing a common control for the assays wherein the control does not cross-react with itself or with reagents for conducting the assays. In a particular embodiment the common control comprises an aqueous medium, a reagent for assessing fibrinogen biological activity and a reagent for assessing platelet count reagent activity.

Another embodiment of the present invention is a method for conducting an assay for platelet function activity and an assay for platelet count on a blood sample containing platelets. Platelet function activity of the platelets is determined along with a control value utilizing a first aliquot of an aqueous medium comprising an antibody for fibrinogen and fixed platelets substantially free of fibrinogen binding sites. The control value is determined concomitantly with, or prior to or after, the step of determining platelet function activity. The platelet count of the sample is also determined along with a control value with utilizing a second aliquot of the above aqueous medium. The control value is determined concomitantly with, or prior to or after, the step of determining platelet count. The above method may be applied to determine one or both of platelet function activity of the platelets and platelet count of the sample.

Another embodiment of the present invention is a kit comprising in packaged combination (a) an aqueous medium comprising an antibody for fibrinogen and fixed platelets substantially free of fibrinogen binding sites, (b) one or more reagents for conducting an assay for platelet function activity and (c) one or more reagents for conducting an assay for platelet count.

### BRIEF DESCRIPTION OF THE DRAWINGS

FIG. 1 is a graph depicting the results of an experiment showing the rate of 4A5-induced aggregation of fibrinogen-coated beads.

FIG. 2 is a graph depicting the results of an experiment showing a correlation between whole blood platelet function assay and 4A5 particle-induced agglutination.

FIG. 3 is a graph depicting the results of an experiment involving the agglutination of fibrinogen coated beads with 4A5-particles.

FIG. 4 is a graph depicting the results of an experiment involving the agglutination of 4A5-particles with fixed platelets.

FIG. 5 is a graph depicting the results of an experiment involving the binding of FITC anti-fibrinogen antibodies to fixed platelets.

FIG. 6 is a graph depicting the results of an experiment involving the agglutination of fixed platelets with anti-fibrinogen particles.

FIG. 7 is a graph depicting the results showing the effect of soluble antibody on agglutination of 4A5-particles with fixed platelets.

FIG. 8 is a graph depicting the results of an experiment involving the rate of agglutination of fixed platelets with 4A5 -particles.

FIG. 9 is a graph depicting the results of an experiment involving the extent of agglutination of fixed platelets with 4A5-particles.

FIG. 10 is a graph depicting the results of an experiment involving the agglutination of fixed platelets with 6D1-particles.

### DETAILED DESCRIPTION OF THE INVENTION

The present invention provides reagents for conducting controls in platelet function and platelet count assays. The control reactions utilize a composition comprising an aqueous medium, a reagent for assessing fibrinogen biological activity and a reagent for assessing platelet count reagent activity. The present invention uses a common control for the assays wherein the control does not cross-react with itself or with reagents for conducting the assays. The composition may further contain stabilizers, emulsifying agents such as detergents and the like, particles, soluble components such as, e.g., proteins and the like, etc., to achieve calibration of assay results.

3. Definitions

Before proceeding further with a detailed description of the present invention, a number of terms as used herein are defined.

Sample—any solution, synthetic or natural, containing platelets, including body fluids such as, for example, whole blood, platelet-containing blood fractions such as plasma, and the like. The amount of the sample depends on the nature of the sample. For fluid samples such as whole blood, the amount of the sample is usually about 30 μl to 3000 μl, preferably, about 2000 μl. The term “sample” includes unprocessed samples directly from a patient or samples that have been pretreated and prepared in any convenient liquid medium although an aqueous medium is preferred.

Associated with—a molecule may be associated with a matrix such as a particle by being non-specifically bound or specifically bound to the matrix, by being physically adsorbed on the surface of the matrix or by being dissolved in the matrix. Non-specific binding of a molecule to a matrix may be achieved by covalently bonding or attaching the molecule to the matrix.

Matrix—a support comprised of an organic or inorganic, solid or fluid, water insoluble material, which may be transparent or partially transparent. The matrix can have any of a number of shapes, such as particle, including bead, film, membrane, tube, well, strip, rod, and the like. The surface of the matrix is, preferably, hydrophilic or capable of being rendered hydrophilic. The body of the matrix is, preferably, hydrophobic. The matrix may be suspendable in the medium in which it is employed. Examples of suspendable matrices are polymeric materials such as latex, lipid bilayers, oil droplets, cells and hydrogels. Other matrix compositions include polymers, such as nitrocellulose, cellulose acetate, poly (vinyl chloride), polyacrylamide, polyacrylate, polyethylene, polypropylene, poly(4 methylbutene), polystyrene, polymethacrylate, poly(ethylene terephthalate), nylon, poly(vinyl butyrate), polysaccharides such as dextrans and modified dextrans, etc.; either used by themselves or in conjunction with other materials.

Binding of molecules such as antibodies to the matrix may be direct or indirect, covalent or non-covalent and can be accomplished by well known techniques, commonly available in the literature. See, for example, “Immobilized Enzymes,” Ichiro Chibata, Halsted Press, New York (1978) and Cuatrecasas, J. Biol. Chem., 245:3059 (1970).

The surface of the matrix may be polyfunctional or be capable of being polyfinctionalized or be capable of binding to a molecule, or the like, through covalent or specific or non specific non covalent interactions. Such binding is indirect where non-covalant interactions are used and is direct where covalent interactions are employed. A wide variety of functional groups are available or can be incorporated. Functional groups include carboxylic acids, aldehydes, amino groups, cyano groups, ethylene groups, hydroxyl groups, mercapto groups and the like. The manner of linking a wide variety of compounds to surfaces is well known and is amply illustrated in the literature (see above). The length of a linking group to the molecule may vary widely, depending upon the nature of the molecule, the effect of the distance between the molecule and the surface on the specific binding properties and the like.

Particles—particles of at least about 0. 1 microns and not more than about 10 microns, usually at least about 1 micron and less than about 6 microns. The particles can be virtually any shape, but are generally spherical with uniform diameters. The particle may have any density, but preferably of a density approximating water, generally fiom about 0.7 to about 1.5g/ml. The particles may or may not have a charge on the surface, either positive or negative, preferably negative. The particles may be solid (e.g., comprised of organic and inorganic polymers or latex), oil droplets (e.g., hydrocarbon, fluorocarbon, silicon fluid), or vesicles (e.g., synthetic such as phospholipid or natural such as cells and organelles).

The solid particles are normally polymers, either addition or condensation polymers, which are readily dispersible in the liquid medium. The solid particles will also be adsorptive or functionalizable so as to bind or attach at their surface, either directly or indirectly, as discussed above for the matrix.

The solid particles can be comprised of polystyrene, polyacrylamide, homopolymers and copolymers of derivatives of acrylate and methacrylate, particularly esters and amides, silicones and the like.

Antibody—an immunoglobulin that specifically binds to and is thereby defined as complementary with a particular spatial and polar organization of another molecule such as a platelet cell surface glycoprotein. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies.

Antibodies may include a complete imnunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affmity for a particular molecule is maintained.

Antiserum containing antibodies (polyclonal) is obtained by well-established techniques involving immunization of an animal, such as a rabbit, guinea pig, goat or the like with an appropriate immunogen and obtaining antisera from the blood of the immunized animal after an appropriate waiting period. State-of-the-art reviews are provided by Parker, “Radioimmunoassay of Biologically Active Compounds,” Prentice-Hall (Englewood Cliffs, N.J., U.S., 1976); Butler, J. Immunol. Meth. 7: 1-24 (1975); Broughton and Strong, Clin. Chem. 22: 726-732 (1976); and Playfair, et al., Br. Med. Bull. 30: 24-31 (1974).

Antibodies can also be obtained by somatic cell hybridization techniques, such antibodies being commonly referred to as monoclonal antibodies. Monoclonal antibodies may be produced according to the standard techniques of Kohler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). Samples of an appropriate immunogen preparation are injected into an animal such as a mouse and, after a sufficient time, the animal is sacrificed and spleen cells obtained. Alternatively, the spleen cells of a non-immunized animal can be sensitized to the immunogen in vitro. The spleen cell chromosomes encoding the base sequences for the desired immunoglobulins can be compressed by fusing the spleen cells, generally in the presence of a non-ionic detergent, for example, polyethylene glycol, with a myeloma cell line. The resulting cells, which include fused hybridomas, are allowed to grow in a selective medium, such as HAT-medium, and the surviving immortalized cells are grown in such medium using limiting dilution conditions. The cells are grown in a suitable container, e.g., microtiter wells, and the supernatant is screened for monoclonal antibodies having the desired specificity.

Various techniques exist for enhancing yields of monoclonal antibodies, such as injection of the hybridoma cells into the peritoneal cavity of a mammalian host, which accepts the cells, and harvesting the ascites fluid. Where an insufficient amount of the monoclonal antibody collects in the ascites fluid, the antibody is harvested from the blood of the host. Alternatively, the cell producing the desired antibody can be grown in a hollow fiber cell culture device or a spinner flask device, both of which are well known in the art. Various conventional ways exist for isolation and purification of the monoclonal antibodies from other proteins and other contaminants (see Köhler and Milstein, supra).

In another approach for the preparation of antibodies the sequence coding for antibody binding sites can be excised from the chromosome DNA and inserted into a cloning vector which can be expressed in bacteria to produce recombinant proteins having the corresponding antibody binding sites.

In general, antibodies can be purified by known techniques such as chromatography, e.g., Protein A chromatography, Protein G chromatography, DEAE chromatography, ABx chromatography, and the like, filtration, and so forth.

4. Specific Embodiments

The method of the invention may be employed in conjunction with an assay for platelet function such as the rapid platelet function assay of U.S. Pat. No. 5,763,199 ('199 patent) and with an assay for platelet count such as that described in U.S. Pat. application Ser. No. 09/177,884 filed Oct. 23, 1998 (the '884 application), the relevant disclosures of which are incorporated herein by reference. The controls contemplated by the present invention may serve as a check for reagent viability or as a calibrator or both.

In one aspect of the present invention, the result of the platelet count and platelet function assays is compared to a control used as a calibrator, which may be performed concomitantly with, or have been performed previously or may be performed after, one or both of the above assays. Samples having known amounts of the respective component of interest may be prepared and performed in the assay and the results charted so as to be able to translate the measurement obtained with the sample to the standard.

In another aspect of the present invention, controls are used to assess the viability of reagents in an assay or variation of the base value depending on the source of the sample. The control utilized for the above assays is a common control and employs a composition comprising an aqueous medium, a reagent for assessing fibrinogen biological activity and a reagent for determining platelet count reagent activity. The controls of the present invention confirm the viability of the reagents used in the platelet count and platelet function assays utilizing the direct biological counterparts involved in those assays. The controls may be carried out concomitantly with, or prior to or after, the platelet function and the platelet count assays. In practice, for example, the control may be carried out one or more days or weeks in advance of subsequent platelet function assays and platelet count assays conducted from one set of reagents in a kit.

The reagent for assessing fibrinogen biological activity may be, for example, antibody for fibrinogen, fibrinogen clotting enzymes and precursors and activators thereof such as, e.g., thrombin, thrombin-like enzymes, thrombin precursors such as prothrombin, activators of prothrombin to thrombin such as, e.g., ecarin, immobilized platelet membranes, Staphylococcus aureus clumping factors (see, e.g., McDevitt, et al., Eur. J. Biochem. (1997) 247(1):416-424), fibrinogen binding peptides, purified GPIIb/IIIa receptor and so forth.

Preferably, the reagent is an antibody for fibrinogen, either polyclonal or monoclonal. Preferably, the antibody for fibrinogen is against regions on the fibrinogen platelet binding sites. More preferably, the antibody is against the C-terminus of fibrinogen (residues g 400 to 411). GPIIb/IIIa is a platelet integrin whose interaction with fibrinogen has been extensively studied and described in the literature. The location of the main IIb/IIIa binding site on fibrinogen has been mapped to the residues g 400 411. One convenient method for obtaining purified antibody, either polyclonal or monoclonal, is to employ the g 400-411 dodecapeptide sequence bound to a matrix. Antibody binding to the sequence on the matrix may then be separated from unbound materials and then released from the matrix or not as desired.

Monoclonal antibodies in the present composition may be monoclonal antibodies directed against the fibrinogen gamma-chain carboxyl-terminus, e.g., 4A5, 11A9, monoclonal antibodies against the D domain of fibrinogen such as, e.g., FD4-7B3 and FD4-4E1 (both from Accurate, Westbury N.Y.), AD1-3 11 and AD 1-313 (both from American Diagnostica, Greenwich Conn.), and the like. Monoclonal antibody 4A5 is a murine monoclonal antibody raised against a peptide sequence in human fibrinogen involved in factor XIII catalyzed cross linking of polymerized fibrin. The antibody reacts with g 400-411 dodecapeptide sequence in fibrinogen as well. In the present invention this antibody, when coupled to a polystyrene bead carrier, will serve as a high fidelity surrogate of GPIIb/IIIa on the platelet surface for use as a calibrator and an external control of fibrinogen in a platelet finction assay. In this embodiment of the present invention, the antibody in the control reagent engages the same site on the fibrinogen molecule that reacts with GPIIb/IIIa, thereby monitoring the state of GPIIb/IIIa activity of fibrinogen. Monoclonal antibody 4A5 is described by Shiba in Am. J. Physiology (1991) 260:C965-C974. Monoclonal antibody 11A9 and 4A5 is described by Matsueda & Bernatowicz in “Characterization of a monoclonal antibody that binds to the carboxyl-terminus of the fibrinogen gamma-chain” in “Fibrinogen 3: biochemistry, biological functions, gene regulation and expression”, pp. 133-136 (1988). M.W. Mosesson et al, editors, Elsevier Science Publishers, New York, N.Y..

The antibody for fibrinogen may be associated with a matrix such as a particle. Attachment of the antibody to the matrix may be achieved by any means known in the art such as discussed hereinabove in the definition of the term “matrix.”

The reagent for assessing the platelet count reagent may be, for example, a reagent for binding an anti-platelet surface receptor antibody. In the case where the reagent for determining platelet count is an antibody against GPIb, then such control reagents may include, for example, purified GPIba, glycocalicin, N-terminal GPIb fragments, fixed platelets, and so forth. Preferably, the reagent is fixed platelets substantially free from fibrinogen antibody binding sites. These sites are those to which an antibody against fibrinogen may bind. By substantially free from such sites means that the fixed platelets either do not have such sites or, if present, the number of such sites is small enough that there is no significant effect on the ability of the reagent to provide an accurate control composition. Other reagents include GPIba receptor or the use of a recombinant, soluble GPIba fragment that is recognized by a anti-GPIba monoclonal antibody, e.g., 6D 1, cultured HEL cells that expresses GPlba on the cell surface, and the like.

Preferably, the medium for carrying out the control methods in accordance with the present invention is an aqueous medium and has properties similar to that for conducting the platelet count and platelet function assays. Other polar cosolvents may also be employed in the medium, usually oxygenated organic solvents of from 1-6, more usually from 1-4 carbon atoms, including alcohols, ethers and the like. Usually, such cosolvents are present in less than about 70 weight percent, more usually, in less than about 30 weight percent. Additionally, various ancillary materials are frequently employed in the method in accordance with the present invention. For example, buffers are normally present in the assay medium, as well as stabilizers for the assay medium and the assay components; surfactants, particularly non-ionic surfactants; binding enhancers, e.g., polyalkylene glycols; or the like.

The pH for the medium is usually in the range of about 2 to about 11, preferably, about 4 to about 9. Various buffers may be used to achieve the desired pH and maintain the pH during the method. Illustrative buffers include HEPES, borate, phosphate, carbonate, Tris, barbital, and the like. The particular buffer employed is not critical to the method but one buffer may be preferred over others in certain circumstances. In some circumstances HEPES is preferred and is present at a concentration of about 0.05M to about 0.001M but generally at a concentration of about 0.01M.

The concentration in the aqueous medium of the reagent for assessing fibrinogen biological activity is usually about 0.1 to about 10 U (units) per ml, more usually, about 0.5 to about 3 U per ml for thrombin control and between 0.01% and 1% solids for particle-associated anti-fibrinogen antibody control. The concentration in the aqueous medium of the reagent for assessing platelet count reagent activity is usually about 10,000 to about 600,000, more usually, about 50 to about 450,000 of fixed platelets per microliter.

It is also within the purview of the present invention to include in the composition agents for adjusting the fluid characteristics of the composition such as, for example, light scattering, absorption, viscosity, and the like. A composition having the appropriate fluid characteristics is achieved by including in the composition particles such as fixed red blood cells, polystyrene microparticles, carbon sol, and the like. In general, the fluid characteristics are adjusted to achieve one or more of light scattering, absorption and viscosity properties of blood under the conditions of the assay such as at or near the isobestic point of hemoglobin. Accordingly, particles such as latex particles and the like having a diameter of about 0.1 to 3 microns are included in the medium to achieve such an effect. The particles may be present in an amount of about 0.1 to about 2 %, usually, about 0.6 to about 1% weight to volume. It is within the purview of the present invention to include more than one particle in the medium. Such particles may be, for example, carbon sol and the like. The amount of additional particles is dependent on the nature and amount of the particles used. In general, the amount of particles such as carbon sol is about 0.001 to about 0.2 %, usually, about 0.003 to about 0.005 % where the carbon sol is a second particle in the medium. A detergent may also be present. Detergents include, for example, Tween 20®, Pluronic A21®, Tetronic®, and so forth. When platelets are present, the detergent should be compatible with the platelets such as, e.g., Pluronic A21. The detergent is usually present in an amount of about 0.01 to 0.5 % weight to volume. The medium may also comprise a polymer such as, for example, polyvinylpyrrolidinone, polyvinylalcohol and the like. The polymer may be present in an amount of about 0.5 to about 5 %, usually, about 1 to about 2 %. The medium may also comprise a protein such as, for example, bovine serum albumin, casein, gelatin, and so forth. The protein may be present in an amount of about 1 to 10 mg/ml.

In one embodiment the fixed platelets of the present composition are pretreated with a modifying agent to specifically modify the fibrinogen antibody binding sites to render the fixed platelets substantially free of fibrinogen antibody binding sites. The modifying agent is capable of removing the fibrinogen antibody binding sites or is capable of rendering such site unviable. The modifying agent may be selected from the groups consisting of blocking agents to block fibrinogen antibody binding sites, digestion agents to specifically digest fibrinogen antibody binding sites, agents for inhibiting the expression of fibrinogen antibody binding sites, and the like.

The blocking agents for blocking fibrinogen antibody binding sites include, for example, an antibody for fibrinogen, and so forth. Conveniently, the antibody for fibrinogen may be the same as the antibody for fibrinogen that forms part of the present control composition. The antibody for fibrinogen may be a soluble antibody or it may be attached to a matrix such as a particle, e.g., liposome, polymeric particle, and so forth. The attachment may be non-covalent or passive, i.e., direct passive, secondary antibody, etc., or it may be covalent. The antibody may be attached by any means known in the art such as discussed hereinabove in the discussion concerning the matrix. In a specific embodiment the antibody is attached covalently by an agent such as paraformaldehyde; a homobifinctional agent such as, e.g., bis(sulfosuccinimidyl) suberate (BS3), disuccinimidyl suberate (DSS), and the like; a heterobifunctional agent such as, e.g., succinimidyl-4-(N-maleimidomethyl)cyclohexane-1-carboxylate (SMCC), sulfosuccinimidyl-4-(N-maleimidomethyl)cyclohexane-1-carboxylate (SMCC), and the like. Such reagents are known in the art and a general discussion concerning the conditions for attaching an antibody may be found, e.g., in literature from the manufacturer of the reagents such as the manufacturer's catalog, in “Bioconjugate Techniques,” Greg T. Hermanson, Pierce Chemical Company (1996), p 140, Academic Press, New York, N.Y., and so forth. The amount of blocking agent used should be sufficient to block substantially all of the fibrinogen antibody binding sites so that the fixed platelets can perform sufficiently in the control reagent. The amount used depends on the nature of the blocking agent, the number of fibrinogen antibody binding sites on the fixed platelets, and the like. In general, the amount of blocking agent is about 0.01 to about 10 micrograms per 108 platelets, usually, about 0.1 to about 1 microgram per 108 platelets.

The digestion agents to specifically digest fibrinogen antibody binding sites include, for example, an enzyme that removes part of the fibrinogen antibody binding site without removing GPIb such as, for example, an enzyme that removes the carboxy terminal group, e.g., a carboxypeptidase, e.g., carboxypeptidase y (CPY), carboxypeptidase A (CPA), carboxypeptidase w (CPW), carboxypeptidase B (CPB), etc., and the like. The amount of the digestion agent used depends on the nature of the digestion agent, the number of fibrinogen antibody binding sites on the fixed platelets, the nature of the epitope to which the antibody binds, and the like. In general, the amount of digestion agent is about 1 to about 100, usually, about 15 to about 35, micrograms per 108 platelets.

The digestion reaction is carried out, for example, by washing fixed platelets in a buffered aqueous medium such as an acetate buffer at a pH of about 4 to 7, usually, about 5 to 6, more usually, about 5.5. The number of platelets is adjusted to 200,000 to about 500,000, usually, about 300,000 to about 400,000, more usually, about 350,000 per microliter using the same buffer. The digestion agent is added and the medium is held at a temperature of about 10 to about 50° C, usually, about room temperature, for a period sufficient to digest the fibrinogen antibody bonding sites. Usually, this period is about 5 minutes to about 24 hours. Then, the digestion agent is deactivated such as by addition of an esterase or protease inhibitor, e.g., phenyl methyl sulfonyl fluoride (PMSF), and the like. The platelets treated in this manner conveniently may be stored in the same buffer.

In another approach fixed platelets may be pretreated with an agent for inhibiting the expression of fibrinogen antibody binding sites. Such agents include, for example, platelet aggregation inhibitors, dipyridomole, cortisol, aspirin, prostaglandin El, adenosine, theophylline and the like. Such reagents are discussed by Weiss in “Antiplatelet Drugs and Therapy,” Williams Hematology, Fifth Edition, pp 1550-1562 (1995) McGraw Hill, New York, N.Y..

The conditions for conducting pretreatment of the fixed platelets using an agent for inhibiting the expression of fibrinogen antibody binding sites are set forth in the above references. Briefly, whole blood is collected into an aqueous medium containing the agent for inhibiting expression of fibrinogen binding sites. Blood may be collected into blood collection tubes containing a medium with the agent. For example, blood collection tubes are available from Becton Dickinson (Franklin Lakes, N.J.) where the tubes contain a citrated medium comprising theophylline, dipyridomole and adenosine. All wash steps and fixation steps are conducted in the present of the agent. The amount of the agent used depends on the nature of the reagent and the like. In general, the amount of digestion agent is about 0.1 to about 50 mg/ml, usually, about 0.5 to 1.5 mg/ml, except for aspirin which may be present in an amount of about 1 to about 500 mg/ml, usually, about 90 to 110 mg/ml.

In one aspect of the present invention an assay for platelet function activity and an assay for platelet count are carried out on a blood sample containing platelets. Platelet function activity of the platelets is determined by a method comprising combining the sample with an aggregating system comprising fibrinogen associated particles to form an assay medium and determining the agglutination of the particles. The agglutination of the particles is related to platelet function activity. A control value is determined with respect to that obtained in the determination of platelet function activity by a method comprising combining fibrinogen associated particles with an aliquot of an aqueous medium comprising an agent for assessing fibrinogen biologic activity and fixed platelets substantially free of fibrinogen binding sites. The concentration of fibrinogen associated particles is comparable to the concentration thereof in the determination of platelet function activity. The agglutination of the particles is determined and is indicative of a base value without the participation of platelets. The control value is determined concomitantly with, or prior to or after, the determination of platelet function activity. The platelet count of the sample is determined by a method comprising subjecting to agglutination conditions a liquid medium comprising the sample and a reagent for agglutinating the platelets and determining the agglutination of the platelets, the agglutination being related to the number of platelets in the sample. A control value is determined with respect to that obtained in the determination of platelet count by a method comprising combining an aliquot of the aqueous medium in the determination of platelet count with the reagent for agglutinating in the determination of platelet count. The concentration of the reagent is comparable to the concentration thereof in the determination of platelet count. The agglutination of the fixed platelets is determined and is indicative of a base value without the participation of platelets from the sample. The control value is determined concomitantly with, or prior to or after, the determination of platelet count.

In another aspect of the present invention an assay for platelet function activity and an assay for platelet count are conducted on a blood sample containing platelets. The platelet function activity of the platelets is determined by a method comprising combining the sample with an aggregating system comprising fibrinogen bound particles that absorb light in the infrared to form an assay medium, irradiating the medium with light in the infrared region, and determining the transmission of infrared light from the assay mixture. The level of transmission is related to platelet function activity. A control value is determined with respect to that obtained in the determination of platelet function activity by a method comprising combining fibrinogen bound particles that absorb light in the infrared with a first aliquot of an aqueous medium comprising a reagent for assessing fibrinogen biological activity and fixed platelets pretreated with a blocking agent for blocking fibrinogen binding sites. The concentration of fibrinogen bound particles is comparable to the concentration thereof in the determination of platelet function activity. The medium is irradiated with light in the infrared region and the transmission of infrared light from the medium is determined and is related to a base value without the participation of platelets.

The control value is determined concomitantly with, or prior to or after, the determination of platelet function activity. The platelet count of the sample is determined by a method comprising subjecting to agglutination conditions a liquid medium comprising the sample and reagent comprising a matrix with which is associated a binding molecule for a platelet cell surface glycoprotein receptor. The extent of agglutination of the platelets is determined and is related to the number of platelets in the sample. A control value is determined with respect to that obtained in the determination of platelet count by a method comprising combining an aliquot of the aqueous medium in the determination of platelet count with a reagent comprising a matrix with which is associated a binding molecule for a platelet cell surface glycoprotein receptor. The concentration of the reagent is comparable to the concentration thereof in the determination of platelet count. The extent of agglutination of the fixed platelets is determined and is indicative of a base value without the participation of platelets from the sample, the control value being determined concomitantly with, or prior to or after, the determination of platelet count.

In another aspect of the present invention an assay for platelet function activity is carried out on a blood sample containing platelets. Platelet function activity of the platelets is determined by a method comprising combining the sample with an aggregating system comprising fibrinogen bound particles that absorb light in the infrared to form an assay medium, irradiating the medium with light in the infrared region and determining the transmission of infrared light from the assay mixture. The level of transmission of infrared light is related to platelet function activity. A control value is determined with respect to that obtained in the determination of platelet function activity by a method comprising combining fibrinogen bound particles that absorb light in the infrared with an aqueous medium comprising a monoclonal antibody for fibrinogen or other agent for assessing fibrinogen biological activity and fixed platelets substantially free of fibrinogen antibody binding sites. The concentration of fibrinogen bound particles is comparable to the concentration thereof in the determination of platelet function activity. The aqueous medium simulates the biological characteristics of whole blood. The medium is irradiated with light in the infrared region. The transmission of infrared light from the medium is determined and the level of transmission is related to a base value without the participation of platelets. In another aspect of the present invention an assay for platelet count is conducted on a blood sample containing platelets. The platelet count of the sample is determined by a method comprising subjecting to agglutination conditions a liquid medium comprising the sample and reagent comprising a matrix with which is associated a binding molecule for a platelet cell surface glycoprotein receptor. The extent of agglutination of the platelets is determined and is related to the number of platelets in the sample. A control value is determined with respect to that obtained in the determination of platelet count by a method comprising combining an aqueous medium comprising fixed platelets substantially free of fibrinogen binding sites with a reagent comprising a matrix with which is associated a binding molecule for a platelet cell surface glycoprotein receptor. The concentration of the reagent is comparable to the concentration thereof in the determination of platelet count. The extent of agglutination of the fixed platelets is determined and is indicative of a base value without the participation of platelets from the sample.

As mentioned above, the platelet count of the sample is determined by a method comprising subjecting to agglutination conditions a liquid medium comprising the sample and a reagent for agglutinating the platelets and determining the agglutination of the platelets, the agglutination being related to the number of platelets in the sample. In one embodiment the platelet count of the sample is determined by a method such as that described in the '884 application. The method comprises subjecting to agglutination conditions a liquid medium comprising the sample and a reagent comprising a matrix with which is associated a binding molecule for a platelet cell surface glycoprotein receptor. The extent of agglutination of the platelets is determined and is related to the number of platelets in the sample.

In the above method platelets are enumerated by combining in a liquid medium a sample suspected of containing platelets with a matrix with which the binding molecule for the platelet cell surface glycoprotein receptor is associated. The matrix is preferably a particle. In certain embodiments the composition of the particle may be any convenient composition, such as Bioglas®, organic polymers, e.g., polyacrylonitrile, polystyrene, polycarbonate, polymethacrylate, combinations thereof, or the like, or other material which absorbs in the infrared or can be made to do so with infrared absorbing dyes as set forth in more detail hereinbelow. The particles are preferably colored to render the results of the agglutination reaction easier to interpret. In a preferred embodiment, the particles are adapted to absorb light in the infrared region (IR).

Preferably, the medium for carrying out the methods herein is an aqueous medium as discussed above. The medium may contain one or more buffers. A suitable buffer also maintains the salt concentration of the liquid within a range suitable for agglutination. Thus, the buffer may contain a concentration of one or more salts such as sodium chloride that maintain the electrolytic balance of the blood within a range suitable for agglutination. Suitable concentrations of sodium chloride in the buffer are between about 0.10M and about 0.20M, typically about 0.15M. Other salts that may be present include, but are not limited to, calcium chloride and magnesium chloride.

In the platelet count assay the medium is subjected to agglutination conditions. Moderate temperatures are normally employed for carrying out the method. The temperature may be constant or may vary. Usually, a constant temperature is employed during the reaction step. The temperature employed is usually about 10 to about 80° C, more usually, about 15 to about 45° C, preferably, the temperature should be at least 25° C, more preferably in the range of about 30 to about 40° C, usually about 37° C.

The number of platelets to be determined usually varies between about 100,000/μl to about 400,000/μl. The amount of the sample employed varies from about 0.05 to about 0.5 ml. The concentration of the matrix with the binding molecule is usually about 0.01 to about 1.0 % solids, more usually, from about 0.2 to about 0.6 % solids. The binding molecule is employed in a concentration of about 10⁻⁷ to about 10⁻⁹ M, more usually, from about 10⁻⁸ M. In many instances the final concentration of each of the reagents is determined empirically to optimize the sensitivity of the method over the range of interest for the suspected platelet concentration.

The order of addition of the various reagents may be varied. Generally, the sample is combined with the liquid medium and the matrix is then added. However, the sample and the matrix may be combined with the liquid medium substantially simultaneously.

The time period for carrying out the assay for platelet count is generally from about 30 seconds to about 1 hour, usually from about 10 seconds to about 2 minutes, more usually, about 20 seconds to about 1 minute. Basically, the time period for the reaction is sufficient to permit substantial agglutination of the matrix. During the period of the reaction, the medium may be agitated manually or mechanically to facilitate binding of the binding molecule to the receptor. Agitation is preferably supplied mechanically, for example, by placing the reaction vessel on a rocker.

Next, the agglutination of the matrix is measured as an indication of the number of receptors present in the medium. The presence of agglutination may be determined visually by observing clumping of the matrix, which would indicate agglutination. Optionally, as mentioned above, the matrix may be colored to aid in visualizing agglutination or clumping of the matrix. Useful dyes are those that absorb in the infrared. The extent of agglutination may be measured spectrophotometrically, turbidimetrically, nephelometrically, and so forth.

The level of agglutination is an indication of the number of receptors present, which is directly related to the number of platelets in the sample. The level of agglutination may be compared against a standard of known platelet number to determine the number of platelets in the sample. Usually, the result will be compared to a calibrator, which may be performed concomitantly or have been performed previously or may be provided as a standard curve. In accordance with the present invention a control is also carried out to assess reagent viability and so forth.

As mentioned above, platelet function activity of the platelets is determined by a method comprising combining the sample with an aggregating system comprising fibrinogen associated particles to form an assay medium and determining the aggultination of the particles is related to platelet function activity.

In one approach the platelet function activity of the platelets is determined by a method comprising combining the sample with an aggregating system comprising fibrinogen bound particles that absorb light in the infrared to form an assay medium, irradiating the medium with light in the infrared region, and determining the transmission of infrared light from the assay mixture. The level of transmission is related to platelet function activity. Such an assay for platelet function is described in the '199 patent. The assay is based on the principle that fibrinogen coated microparticles exhibit a visible agglutination reaction in whole blood in the presence of activated platelets with normal GPIIb/IIIa receptors. In practice, the assay requires the presence of an agglutination medium, preferably GPIIb/IIa receptor ligand coated microparticles, a platelet activating agent, means for observing the aggregation of the microparticles, and means for recording, compiling, and displaying the results.

A GPIIb/IIIa receptor ligand is a small organic molecule, polypeptide, protein, monoclonal antibody or nucleic acid that binds, complexes or interacts with GPIlb/IIIa receptors on the platelet surface. Platelet mediated aggregation of the microparticles results when the GPIIb/IIIa receptors on the surface of platelets bind, complex or otherwise interact with the GPIIb/IIIa receptor ligands on the particles or beads. Typical GPIIb/IIIa ligands include fibrinogen, monoclonal antibody 10E5 (Coller, et al., J. Clin. Invest. 72:325 (1983)), monoclonal antibody c7E3 (The EPIC Investigators, N.E. Journal of Med., 330:956 (1994)), von Willebrand factor, fibronectin, vitronectin and other ligands that have an arginine glycine-aspartic acid (RGD) sequence or other peptides or peptidomimetics that mimic this sequence (Cook, et al., Drugs of the Future 19:135 (1994)).

Platelet activating agents are those agents that activate platelets, i.e., enable the platelets to carry out their intended biological function. One such agent is a thrombin receptor activator, i.e., a peptide, protein, antibody or small organic molecule that induces platelet activation via the thrombin receptor, i.e., which increases the rate of agglutination when platelets whose GPIIb/IIIa receptors are not blocked when the platelets are combined with a GPIIb/IIIa receptor ligand bound to solid surfaces. A suitable peptide is any peptide of appropriate sequence and size to activate platelets, as described above. The peptide can comprise a thrombin receptor activating agent, thrombin, or a portion thereof, such that the amino acid sequence of the peptide or peptide mimic result in activation of the platelets.

The agglutination for the platelet function assay may involve any suitable solid surface bearing a receptor ligand. Preferably, the surface is a small polymeric bead or microparticle to which a GPIIb/IIIa receptor ligand is covalently bound or absorbed. The polymeric microparticles can be virtually any shape, but are generally spherical with uniform diameters ranging from about 0.1 mm to about 50 mm in diameter. Preferred diameters are from about 1 mm to about 10 mm in diameter, most preferably about 6 mm. For the most part the particle composition with and without the dye will be as described above with respect to the platelet count assay.

Preferably, the concentration of beads is adjusted so that the platelet/bead ratio is from about 1.9 to about 2.8. A GPIIb/IIIa ligand may be covalently or ionically coupled to the bead, or the ligand may be simply coated on the bead.

After the sample has been combined with the reagents, desirably it will be heated to a temperature above room temperature, but below interference with the assay, so as to insure that the temperature can be controlled without adversely affecting the assay result. Desirably, the temperature should be at least 25°, preferably in the range of 30-40° C, more preferably about 37° C. The reaction medium is usually agitated during the period of the reaction. The total time of the readings from the zero time (time of mixing), may range from about 10 sec. to 5 min., more usually about 30 sec. to 5 min., and preferably about 30 sec. to 2 min. The data may be analyzed by any convenient means, particularly using an algorithm that can manipulate the data in relation to calibrators and/or controls.

The above assays and control reactions, preferably, may be conducted in a device such as that described in U.S. Pat. application Ser. No. 08/933,443 filed Sep. 18, 1997, in a manner as discussed therein. The disclosure of this application is incorporated herein by reference in its entirety.

Another aspect of the present invention is a kit comprising in packaged combination (a) an aqueous medium comprising an antibody for fibrinogen and fixed platelets substantially free of fibrinogen binding sites, (b) one or more reagents for conducting an assay for platelet function activity and (c) one or more reagents for conducting an assay for platelet count. The kit may also include a sample collection container and/or a device for carrying out the present method and the platelet fimction assay and the platelet count assay. The relative amounts of reagents may be varied widely to provide for concentrations in solution of the reagents that substantially optimize the sensitivity of a determination. Where appropriate, the reagents can be placed in an airtight package in order to maintain the activity of any reagents. The package may be, for example, a bag, pouch, or the like fabricated fiom a material that is substantially non- permeable to moisture. Such materials include, by way of example and not limitation, plastic, aluminum foil, and the like. For blood samples the kit may also include an article for piercing a person's skin, disinfectant or sterilizing pads and so forth. The kit may also include calibrators and standards.

### EXAMPLES

The following examples are offered by way of illustration and not limitation. Parts and percentages are by weight unless otherwise indicated. Temperatures are in degrees Centigrade (° C) unless indicated otherwise. The following preparations and examples illustrate the invention but are not intended to limit its scope.

The following abbreviations are used:

1. FITC—fluorescein isothiocyanate from Sigma.

EDTA—ethylenediaminetetraacetate from Fisher Scientific, Pittsburgh, Pa.

Fg—fibrinogen from Enzyme Research Labs, South Bend, Ind.

F/P—moles of FITC incorporated/ mole of Antibody

BRS—BRS Com, Inc., Winchester, Mass.

Dako—Dako Corporation, Carpenteria, Calif.

Sigma—Sigma Chemical Company, St. Louis, Mo.

NYBC—New York Blood Center.

Biopool—Ventura, Calif.

Costar—Cambridge, Mass.

TBS—Tris-Buffered Saline (50 mM Tris/HCl, pH 7.4, 150 mM NaCl)

11 Navy 9 —platelets with platelet activation inhibitors from Dr. Marjorie Reed, University North Carolina, Chapel Hill, N.C.

12 Navy 58 —platelets with platelet activation inhibitors from Dr. Marjorie Reed.

BSA—bovine serum albumin

mAb—monoclonal antibody

IR—infrared

SEM—standard error of measurement

PSM—polystyrene microparticles

### Example 1

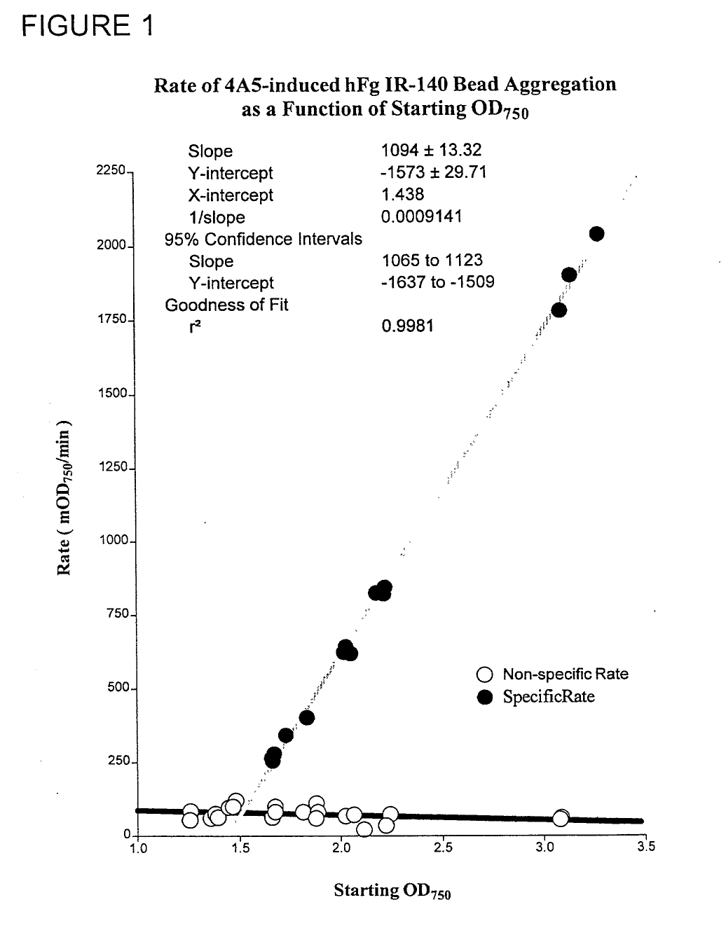

Monoclonal antibody 4A5, directed against the fibrinogen gamma-chain C-terminal dodecapeptide (C12), was conjugated to PSM particles (2 -3 microns in diameter, Spherotech, Libertyville, Ill.) by forming the activated ester using 1-ethyl-3-(3 dimethylaminopropyl)carbodiimide (EDC)/N-hydroxysuccmimide (NHS) as is well-known in the art. See, for example, “Bioconjugate Techniques,” supra. These 4A5-coated particles agglutinated Fg coated, IR dyed latex in a specific manner (FIG. 1). The particular formulation for a control reagent in this example was as follows:

(i) mAb 4A5 covalently coupled to 2 micron polystyrene latex

(ii) light scattering liquid medium wherein the medium contained 0.6-1 % (weight to volume) white latex particles (1 micron), 0.003 % (weight to volume) carbon sol, 0.4 % (weight to volume) detergent and 1 % polyvinylpyrrolidone.

In use the mAb 4A5 coated latex and the light scattering medium were combined together, preferably, right before use in a Vacutainer-like tube, and introduced into an assay cartridge for conducting a platelet function assay in accordance with that disclosed in the '199 patent. The results are shown in FIG. 1, which shows the dependency of the rate of agglutination on the starting optical density (Fg coated bead concentration) and that the reaction is fully inhibited by the excess of g 400 411 synthetic peptide.

The experiments were carried out as follows: The rate of 4A5-coated 2 micron polystyrene latex bead induced agglutination of Fg-coated IR- 140 dyed beads was measured as a function of starting OD at 750 nm (filled circles). Starting OD at 750 nm is linearly proportional to the Fg-coated IR-140 dyed bead concentration and, therefore, can be used instead of it. The concentration of 4A5-coated beads was kept constant at 0.1 % solids. The specificity of the reaction was demonstrated by running the assay in the presence of 0.4 mM gamma C12 peptide (open circles) See FIG. 1.

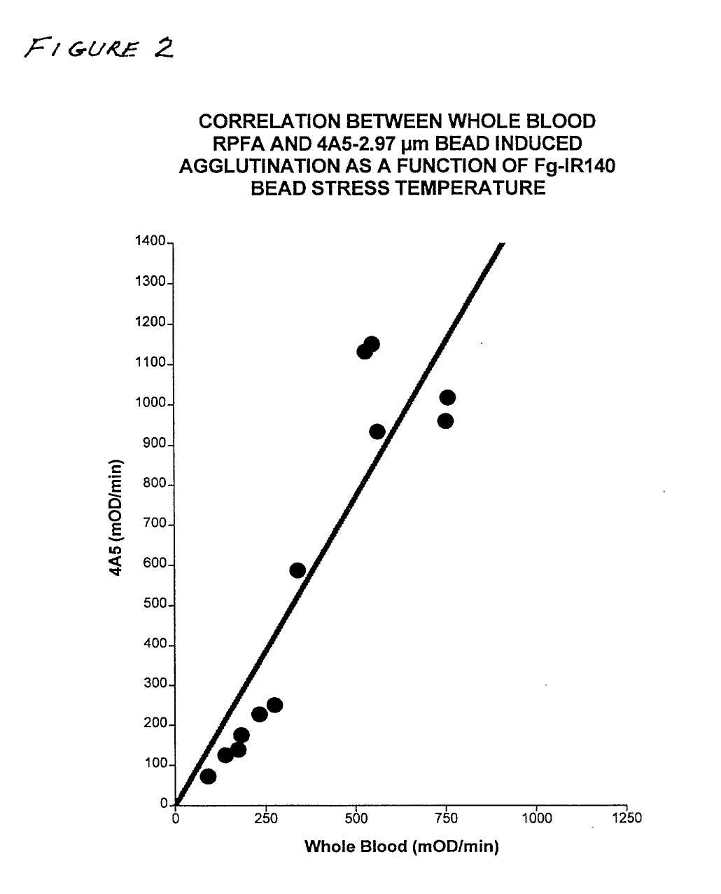

In the above study mAb 4A5 coated particles detected activity loss of Fg IR dyed beads, which was consistent with activity loss observed in whole blood in an rapid platelet function assay conducted as described in the '199 patent. The results are summarized in FIG. 2.

The rate of mAb 4A5 induced agglutination is linearly proportional to the Fg coated, IR dyed bead concentration (FIG. 1).

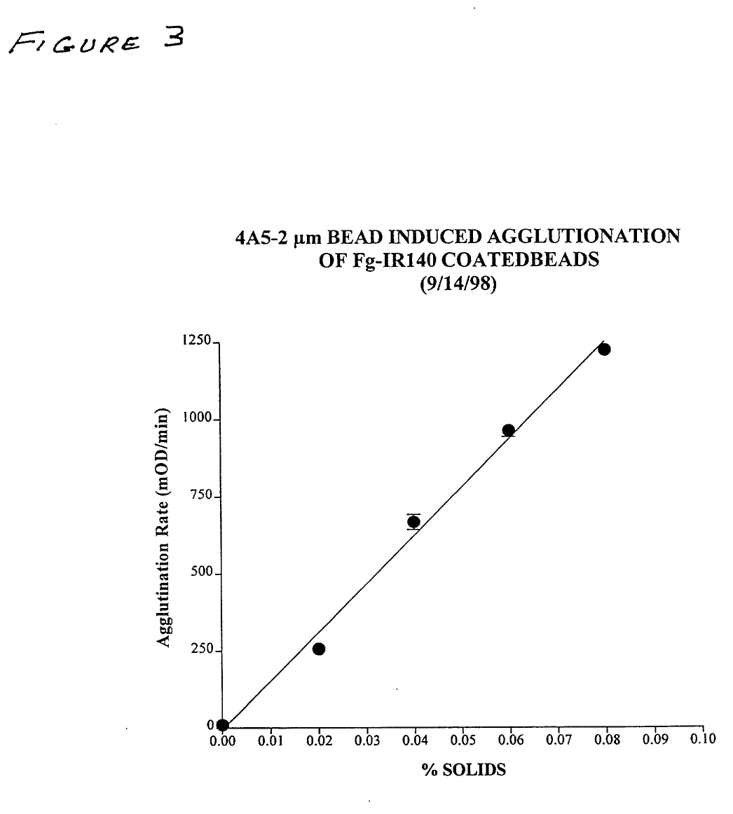

The rate of mAb 4A5 induced agglutination is linearly proportional to the mAb 4A5 coated bead concentration (FIG. 3). These two features, together with an adequate quantitation of the Fg coated, IR dyed bead activity loss, demonstrate the use of the 4A5- based reagent as a calibrator and control for the platelet function assay.

FIG. 2 shows a correlation between whole blood platelet finction and 4A5- 2.97 micron bead induced agglutination as a function of fibrinogen IR140 bead stress temperature.

FIG. 3 shows the agglutination rate (mOD/min) of 4A5 -bead induced agglutination of fibrinogen IR 140 coated beads as a function of 4A5 particle concentration (% solid).

### Example 2

Agglutination of 4A5-particles with fixed platelets was carried out as follows: 0.5% 2.12 micron 4A5 particles were incubated in the presence or absence of 5 mg/ml g 400 411 (C12) for 1 hour at room temperature. 50 ml of about 250,000/ml Biopool fixed platelets in Tris Buffered Saline were placed in triplicate wells of Costar # 3369 microtiter plates. 10 ml of the 4A5-particle control or 4A5 plus C12 mixture was added to the fixed platelets and the reaction was monitored at 360 nm for 10 minutes (FIG. 4). In a separate experiment, 4A5 particles at the same final concentration (0.08%) showed background rates of agglutination of about 4.0.

4A5, 18C6, Serum mouse IgG, Fab′2 anti mouse IgG were FITC-labeled by a procedure similar to that described by Schmidt, et al. J. Biological Chemistry (1998) 273:15061-15068. Other procedures for FITC-labeling are well known in the art.

The various antibodies used are set forth in Table 1.

TABLE 1

| FITC Antibody | Fg Epitope | F/P | Source |
|---|---|---|---|
| 4A5 | g 400 ˜ 11 | 6.9 | BRS |
| Polyclonal Anti Fg | total Fg | 2.3 | Dako |
| Serum mouse | lgG | 7.5 | Sigma |
| 18C6 | Fibrinopeptide B | 5.3 | B. Kudryk (NYBC)* |
| Fab'2 anti-mouse lgG | | 4.2 | Sigma |

*18C6 is also available from Accurate Scientific, Westbury, NY.

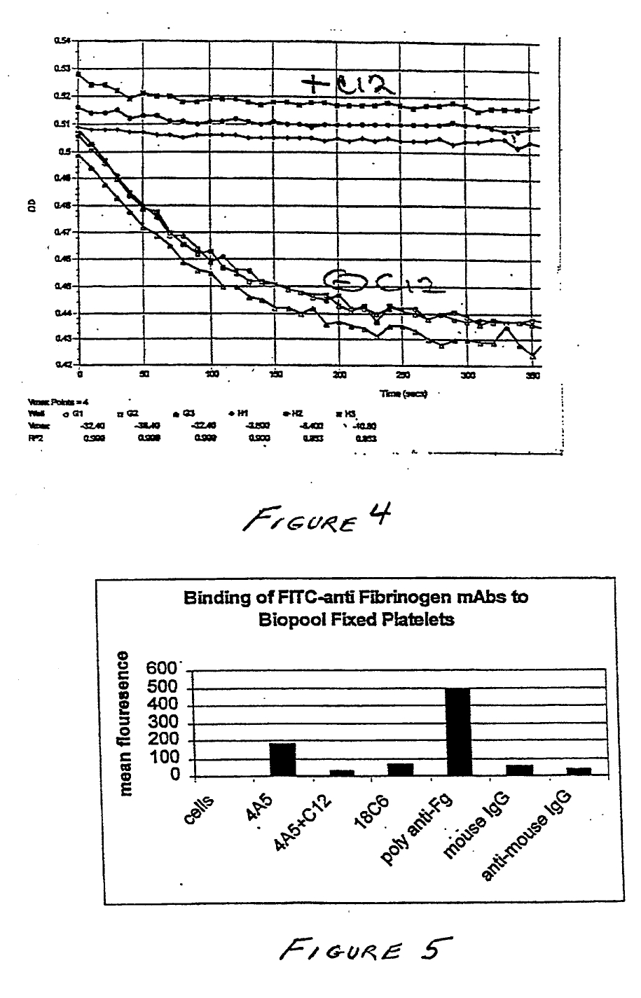

FIG. 4 shows that the interaction between 4A5 particles with fixed platelets resulted from specific binding of 4A5 to fibrinogen that is present on the fixed platelets. This was tested as discussed above in an agglutination reaction in which the 4A5 particles were pre incubated with a peptide (C12) comprising the last 12 residues of the fibrinogen gamma chain, an analog of the 4A5 immunogen and corresponding to the fibrinogen platelet binding site (FIG. 4). Pre incubation with the C 12 peptide at about 1 mg/ml completely abolished the agglutination between 4A5 particles and fixed platelets, which suggested that the interaction was due solely to nascent fibrinogen associated with the fixed platelets. This hypothesis was confirmed by flow cytometry in which soluble FITC labeled 4A5 and polyclonal anti fibrinogen antibodies specifically reacted with the fixed platelets (FIG. 5).

FIG. 5 shows the results of binding of FITC anti-fibrinogen mAb to Biopool fixed platelets. Biopool fixed platelets were diluted to about 50,000/ ml in Tris-Buffered Saline containing about 5 mg/ml of BSA. 10 ml of FITC labeled was added to a final concentration of about 35-40 mg/ml and allowed to incubate for 20 minutes in the dark at ambient temperature. Samples were washed with 1 ml of Tris-Buffered Saline containing 0.05 % sodium azide and analyzed within 30 minutes by Flow Cytometry at Cytometry Research Services, San Diego, Calif.

These results demonstrated that the interaction of 4A5-particles with fixed platelets was due to Fg associated on the platelet surface. It was thus hypothesized that inhibitors of platelet activation could be included during the blood draw and subsequent processing of fixed platelets in order to minimize surface fibrinogen expression resulting from platelet activation. Such platelets would therefore have diminished rates of agglutination when added to 4A5-particles. To test this hypothesis, fixed platelets were prepared under different conditions designed to minimize platelet activation. These platelets (11 Navy 9 and 12 Navy 58) were provided by Dr. Marjorie Reed and Dr. Arthur Brode, University of North Carolina. The preparation was carried out in a manner similar to that described by Reed, et al., PNAS (USA) (1995) 92:397-401. Some of the conditions involved adjusting the paraformaldehyde concentration and time of fixation and/or including prostaglandin E1 at ˜1 μg/ml.

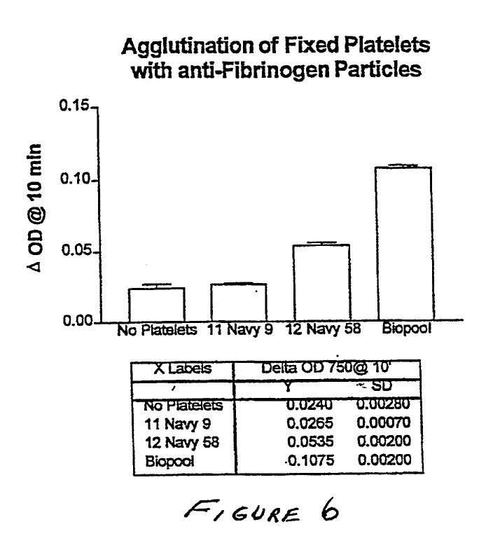

When tested in agglutination experiments carried out as discussed above with 4A5 beads these platelets showed significantly less interaction than Biopool fixed platelets, with values of 0.026 (11 Navy 9) and 0.053 (12 Navy 58). A negative control consisting of 4A5 beads in the absence of platelets gave OD values of 0.024. (FIG. 6). Platelets were diluted to 300,000/ml in TBS and placed in duplicate wells of a microtiter plate. A non specific agglutination control containing TBS with no platelets was also included. 2 mm microspheres coated with a mAb specific for fibrinogen was added and the agglutination was continuously recorded with constant shaking for 10 minutes. The difference in OD at t =0 and t =600 seconds for each sample is shown +1 SEM. Thus, platelets can be prepared having minimal surface expression of fibrinogen.

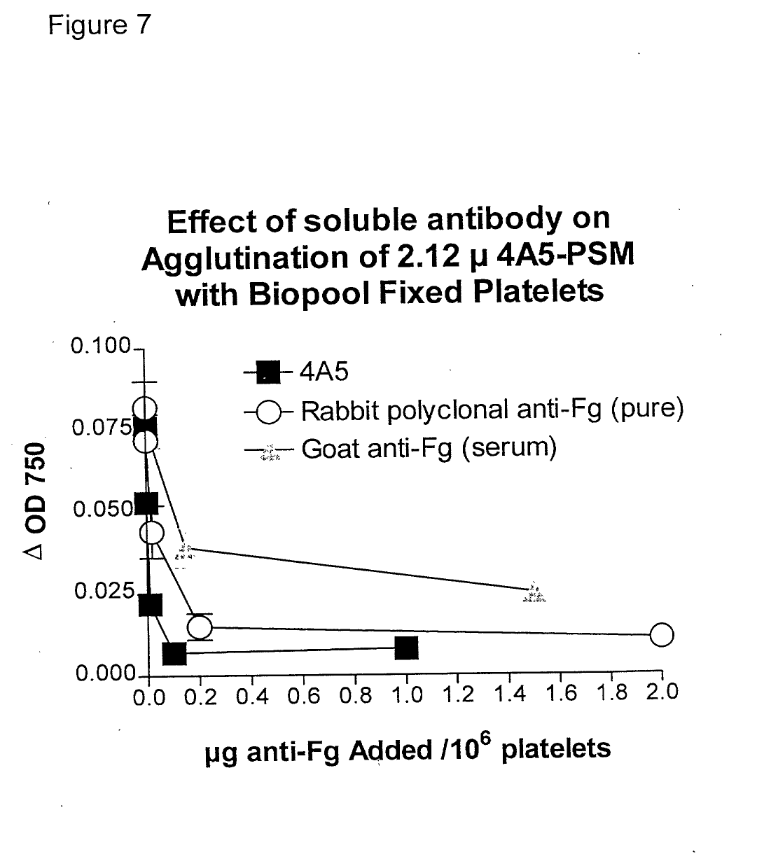

Experiments were then performed to demonstrate that the addition of soluble 4A5 or polyclonal anti-fibrinogen blocked the interaction between 4A5 beads and Biopool fixed platelets (FIG. 7). FIG. 4 shows the effect of soluble antibody on agglutination of 2.12 micron 4A5-PSM with Biopool fixed platelets. These experiments were carried out by adding antibody (0.1 to 20 mg/ml) to 1 mg fixed platelets in TBS/BSA buffer. After 30 minutes at room temperature, unbound antibody was removed by centrifugation. Agglutination experiments were carried out as described above.

The results demonstrated that addition of about 0.1 1.0 mg of 4A5/106. Biopool fixed platelets essentially eliminated the interaction with 4A5 coated beads. Moreover, the results further demonstrate that polyclonal anti fibrinogen can be used as an alternative to monoclonal antibody 4A5.

### Example 3 —Carboxypeptidase Y (CPY) digestion of fixed platelets

Fixed platelets (Biopool, Ventura, Calif.) were washed three times by centrifugation a 3000 ×g for 5 minutes at 4° C in 0.05M sodium acetate buffer, pH 5.5 and adjusted to about 2.5 ×108/ml in the same buffer as judged by electronic particle counting (Coulter, Hialeah, Fla.). Carboxypeptidase Y (Calbiochem, La Jolla, Calif.) was added at about 32 μg of CPY per 1.0 ×108 platelets and allowed to incubate for about 24 hours at room temperature with gentle rocking (Vari-Mix, Thermolyne, Dubuque Iowa). The reaction was terminated with 1 mM PMSF (Sigma). Then, platelets were washed twice by centrifugation in Tris-buffered saline. Control platelets were prepared analogously but in the absence of CPY.

### Example 4 —CPY-digested platelet agglutination with-4A5 particles

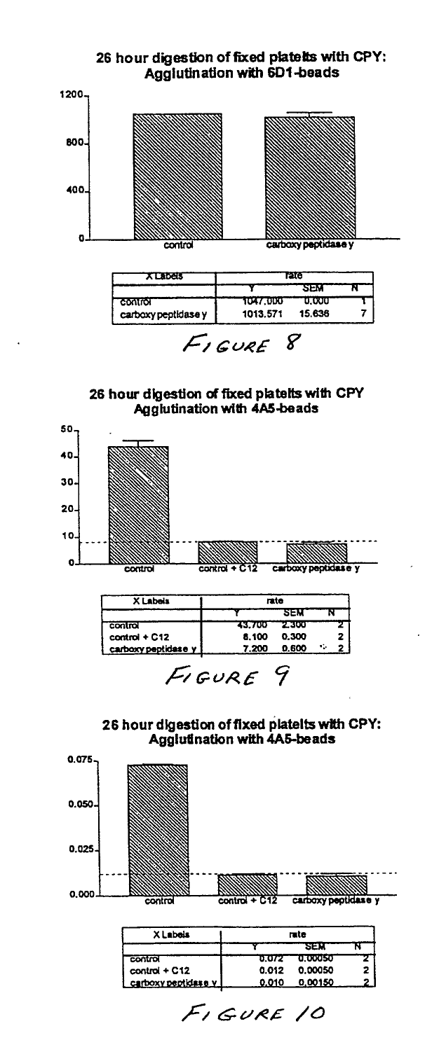

Control (50 ml) or CPY-digested platelets at 1.3 ×108/ml were placed in microtiter wells and 10 ml of 0.5% 4A5 particles in buffer or in buffer containing 0.1 mg/ml C12 peptide was added and the reaction monitored at 750 nm as described. The C12 peptide was added to show the minimum non-specific background rate of agglutination in the system. The results are depicted in FIG. 8 and FIG. 9, and show the rate of agglutination and extent of agglutination, respectively.

### Example 5 —Platelet agglutination with 6D 1 particles

IR-dyed particles (6 micron diameter, IDC, Portland, Oreg.) were coated covalently with anti- GPIba monoclonal antibody 6D 1 by a one-step procedure using EDC coupling as follows:

One ml of 2% IR-dyed particles were centrifuged at 1500 ×g for 5 minutes and resuspended in 0.1 ml (1/10 volume) of deionized and ultra-filtered (DIUF) water. 6D1 monoclonal antibody (from Dr. Barry Coller, Mt. Sinai School of Medicine, New York, N.Y., purified by affmity chromatography on immobilized protein A as described by Scudder, et al., Methods in Enzymology (1992) 215:295-311) was diluted to 0.8 to 0.025 mg/ml in 50 mM MES, pH 5.5, representing a 1:25 to 1:800 weight to weight (w/w) ratio of antibody to polystyrene, respectively. 1 ml of antibody was added to 0.1 ml of the concentrated particles, followed immediately by the addition of 0.1 ml (1/10 volume) of freshly prepared Ethyl-3-(3-Dimethylaminopropyl) carbodiimide Hydrochloride (EDC), obtained from either Pierce Chemical Co., Rockford Ill., or Aldrich Chemical Co., Milwaukee Wis., in DIUF water. The reaction was allowed to proceed for 2 hours at ambient temperature on a platform rocking apparatus (Vari-Mix, Thermolyne, Dubuque Iowa) and then centrifuged as above. The supernatants were saved and the absorbance at 280 nm of the starting antibody solutions and supernatants recorded on a spectrophotometer in order to estimate the amount of protein coupled to the particles. The particle pellets were resuspended in 1 to 3 ml of 10 mM (HEPES)/NaOH, 150 mM NaCl, 0.02% NaN3, pH 7.4 containing 2 mg/ml bovine serum albumin (BSA) (Sigma Chemical Co., St. Louis Mo.) (HBS)/BSA) and washed three times by centrifugation. The particles were suspended to 2% solids in the same buffer and stored at 2-8° C.

The 6D1 particles were diluted to 1.6-1.7 ×108/ml in Tris buffered saline in a 96 well microtiter plate (Costar, Cambridge, Mass.). Control or CPY-digested fixed platelets were added and the rate of agglutination was monitored at 750 nrn on a SpectraMax 340 kinetic plate reader (Molecular Devices, Sunnyvale, Calif.). The results are depicted in FIG. 10 and show a 97% retention of the CPY-digested platelets ability to agglutinate with 6D 1 particles relative to the control.

The results of the above experiments demonstrate that a control using 4A5 particles and fixed platelets is attained successfully by either: 1) limiting the surface expression of platelet fibrinogen by the inclusion of platelet aggregation inhibitors. 2) Blocking residual 4A5 binding sites on fixed platelets with soluble 4A5, and/or 3) Specifically digesting the fibrinogen 4A5 binding site from the surface with a carboxypeptidase that does not affect the GPIba receptor.

It is evident from the above results that a simple method is provided by the present invention for a common control in platelet function assays enumeration of platelets. A whole blood sample may be used and the method can be carried out in conjunction with a rapid platelet finction assay such as that described above in U.S. Pat. No. 5,763,199.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A composition comprising an aqueous medium, a reagent for assessing fibrinogen biological activity and a reagent for assessing platelet count reagent activity.

2. A composition according to claim 1 wherein said reagent for assessing fibrinogen biological activity is selected from the group consisting of an antibody for fibrinogen, fibrinogen clotting enzymes and precursors and activators thereof, imobilized platelet membranes, Staphylococcus aureus clumping factor, fibrinogen binding peptides and purified GPIIb/IIIa receptor.

3. A composition according to claim 1 wherein said reagent for binding a receptor for determining platelet count is a reagent containing GPIb (fixed platelets).

4. A composition according to claim 1 further comprising an agent for adjusting the fluid characteristics of said composition.

5. A composition comprising an aqueous medium, an antibody for fibrinogen and fixed platelets substantially free of fibrinogen antibody binding sites.

6. A composition according to claim 5 further comprising an agent for adjusting the fluid characteristics of said composition.

7. A composition according to claim 5 wherein said antibody for fibrinogen is a monoclonal antibody.

8. A composition according to claim 7 wherein said monoclonal antibody recognizes the g400-411 epitope of fibrinogen.

9. A composition according to claim 5 wherein said antibody recognizes the g400-411 epitope of fibrinogen.

10. A composition according to claim 5 wherein said fixed platelets have been pretreated with a modifying agent to specifically modify said antibody fibrinogen binding sites.

11. A composition according to claim 10 wherein said modifying agent is selected from the groups consisting of blocking agent to block fibrinogen antibody binding sites, digestion agents to specifically digest fibrinogen antibody binding sites and agents for inhibiting the expression of fibrinogen antibody binding sites.

12. A composition according to claim 11 wherein said modifying agent is selected from the group consisting of antibodies for fibrinogen, enzymes and platelet aggregation inhibitors.

13. A composition according to claim 5 wherein said antibody for fibrinogen is associated with a particle.

14. A method for conducting a control for an assay for platelet function activity and an assay for platelet count, said method comprising utilizing a common control for said assays wherein said control does not cross-react with itself or with reagents for conducting said assays.

15. A method according to claim 14 wherein said common control comprises an aqueous medium, a reagent for assessing fibrinogen biological activity and a reagent for assessing platelet count reagent activity.

16. A method according to claim 15 wherein said reagent for assessing fibrinogen biological activity is selected from the group consisting of antibodies for fibrinogen and fibrinogen clotting enzymes.

17. A method according to claim 15 wherein said reagent for binding the reagent for determining platelet count is a reagent containing GPIb.

18. A method according to claim 14 wherein said common control comprises an aqueous medium, an antibody for fibrinogen and fixed platelets substantially free of fibrinogen antibody binding sites.

19. A method according to claim 18 wherein said antibody for fibrinogen is a monoclonal antibody.

20. A method according to claim 19 wherein said monoclonal antibody recognizes the g400-411 epitope of fibrinogen.

21. A method according to claim 18 wherein said antibody for fibrinogen is a polyclonal antibody that recognizes the g400-411 epitope of fibrinogen.

22. A method according to claim 18 wherein said fixed platelets have been pretreated with a modifying agent to specifically modify said antibody fibrinogen binding sites.

23. A method according to claim 22 wherein said modifying agent is selected from the groups consisting of blocking agent to block fibrinogen antibody binding sites, digestion agents to specifically digest fibrinogen antibody binding sites and agents for inhibiting the expression of fibrinogen antibody binding sites.

24. A method according to claim 23 wherein said modifying agent is selected from the group consisting of antibodies for fibrinogen, enzymes and platelet aggregation inhibitors.

25. A method according to claim 18 wherein said antibody for fibrinogen is associated with a particle.

26. A method according to claim 15 wherein said aqueous medium is combined with a light scattering medium.

27. A method for conducting an assay for platelet function activity and an assay for platelet count on a blood sample containing platelets, said method comprising
(a) determining platelet finction activity of said platelets,
(b) determining a control value with respect to that obtained in step (a) utilizing an aliquot of an aqueous medium comprising an antibody for fibrinogen and fixed platelets substantially free of fibrinogen binding sites, said control value being determined concomitantly with step (a) or prior to or after step (a),
(c) determining platelet count of said sample, and
(d) determining a control value with respect to that obtained in step (c) utilizing an aliquot of said aqueous medium of step (b), said control value being determined concomitantly with step (c) or prior to or after step (c).

28. A method according to claim 27 wherein said fixed platelets have been pretreated with a modifying agent to specifically modify said antibody fibrinogen binding sites.

29. A method according to claim 28 wherein said modifying agent is selected from the groups consisting of blocking agent to block fibrinogen antibody binding sites, digestion agents to specifically digest fibrinogen antibody binding sites and agents for inhibiting the expression of fibrinogen antibody binding sites.

30. A method for conducting an assay for platelet function activity on a blood sample containing platelets, said method comprising
(a) determining a value for one or both of platelet function activity of said platelets and platelet count of said sample and
(b) determining a control value with respect to that obtained for each of the values obtained in step (a) utilizing an aqueous medium comprising a monoclonal antibody for fibrinogen and fixed platelets substantially free of fibrinogen binding sites.

31. A method according to claim 30 wherein said fixed platelets have been pretreated by a method selected from the group consisting of treatment with a blocking agent to block fibrinogen binding sites, treatment with a digestion agent to specifically digest fibrinogen binding sites and treatment with an agent for inhibiting the expression of fibrinogen binding sites.

32. A kit comprising in packaged combination:
(a) an aqueous medium comprising an antibody for fibrinogen and fixed platelets substantially free of fibrinogen binding sites,
(b) one or more reagents for conducting an assay for platelet finction activity and
(c) one or more reagents for conducting an assay for platelet count.

33. A kit according to claim 32 wherein said one or more reagents for conducting an assay for platelet finction activity is an aggregating system comprising fibrinogen bound particles that absorb light in the infrared.

34. A kit according to claim 32 wherein said one or more reagents for conducting an assay for platelet count is a reagent comprising a matrix with which is associated a binding molecule for a platelet cell surface glycoprotein receptor.

35. A kit according to claim 32 wherein said fixed platelets have been pretreated by a method selected from the group consisting of treatment with a blocking agent to block fibrinogen binding sites, treatment with a digestion agent to specifically digest fibrinogen binding sites and treatment with an agent for inhibiting the expression of fibrinogen binding sites.
